(19) United States
(12) Patent Application Publication
(10) Pub. No.: US 2004/0234507 A1
(43) Pub. Date: Nov. 25, 2004

(54) Submucosal xenografts

(76) Inventor: Kevin R Stone, Mill Valley, CA (US)

Correspondence Address: Mark G Lappin, McDermott, Will & Emery, 28 State Street, Boston, MA, 02109, US

(21) Appl. No.: 10/476,806

(22) PCT Filed: Apr. 18, 2002
(86) PCT No.: PCT/US02/12295

Related U.S. Application Data
(60) Provisional application No. 60/289,328, filed on May 7, 2001.

(51) Int. Cl. A61K 45/00

(52) U.S. Cl. 424/93.7

(57) ABSTRACT

The invention provides a substantially non-immunogenic submucosal xenograft for implantation into humans. The invention further provides methods for preparing the xenograft by removing at least a portion of a submucosa from a non-human animal; washing the xenograft in saline and alcohol; subjecting the xenograft to cellular disruption treatment; and digesting the xenograft with a glycosidase to remove surface carbohydrate moieties. The invention also provides an article of manufacture produced by the above-identified method. The invention further provides a submucosal xenograft for implantation into a human including a portion of a submucosal tissue from a non-human animal, wherein the portion has substantially no surface carbohydrate moieties that are susceptible to glycosidase digestion. Each of the xenografts has substantially the same mechanical properties as native soft tissue.

### FIELD OF THE INVENTION

The invention relates to the field of surgical repair of injuries, and in particular, to the replacement and repair of defective human tissue using a substantially immunologically compatible submucosa from a non-human animal.

### BACKGROUND OF THE INVENTION

Small intestinal submucosa (“SIS”) is a naturally occurring complex extracellular matrix material. Typically taken from porcine small intestine, the submucosa naturally occurs between the mucosal and muscular layers of the small intestine. SIS does not contain cells; rather this material has a complex organization of collagen that forms a matrix. SIS is primarily protein with secondary amounts of carbohydrates and lipids.

When implanted into a patient's body, SIS is a tissue engineering biomaterial that provides an environment that allows a patient's body to replace and repair damaged tissue. Moreover, this biomaterial combines strength and flexible handling while providing an environment for the growth of the body's own tissue.

While the use of SIS has been suggested for the treatment of partial and full-thickness skin loss injury, urinary incontinence, and repair and reinforcement of soft tissue, there is a need in the art for a tissue engineering biomaterial that is substantially non-immunogenic in primates, particularly humans.

### SUMMARY OF THE INVENTION

The invention provides a substantially non-immunogenic submucosal xenograft for implantation into a primate, particularly a human. The invention also provides methods for processing submucosal xenografts, to produce xenografts with reduced immunogenicity but with substantially native elasticity and load-bearing capabilities. The methods of the invention include cellular disruption treatment and glycosidase digestion of carbohydrate moieties of the xenograft. In addition to or in lieu of these steps, the methods of the invention include the steps of treatment of submucosa with radiation; one or more cycles of freezing and thawing; treatment with a chemical cross-linking agent; and treatment with alcohol or ozonation.

In one embodiment, the invention provides an article of manufacture comprising a small intestinal submucosa (“SIS”) xenograft for implantation into a human. In another embodiment, the invention provides a method of preparing a submucosal xenograft for implantation into a human, which includes removing at least a portion of a SIS from a non-human animal to provide a xenograft; washing the xenograft in water and alcohol and digesting the xenograft with a glycosidase to remove carbohydrate moieties, whereby the xenograft has substantially the same mechanical properties as a portion of a native soft tissue, whereby the xenograft is substantially non-immunogenic in primates, particularly humans.

The submucosal xenograft has significant suture holding strength. Individual sheets can be rolled, folded or shaped into multiple layers for extra strength. The submucosal xenograft can be used for large or chronic dermal injuries. Other possible applications are (a) as an adhesion barrier; (b) corneal use; (c) periodontal use; (d) esophageal use; (e) as an organ patch; (f) as a hemostatic plug; (g) for use in treating cleft palate; (h) other soft tissue repair; or (i) normal wound care.

### DETAILED DESCRIPTION OF THE PREFERRED EMBODIMENTS

Definitions. The term “submucosal tissue”, means a layer of loose connective tissue beneath a mucous membrane, called also the tela submucosa. In particular, submucosal tissue includes the tissue from the small intestines, the stomach, the urinary bladder, and other organs. In an animal, prior to the treatment of the invention, submucosa generally contain blood and lymph vessels, lymph nodes, nerve fibers and elastic fibers.

The term “soft tissue”, as used herein, refers to cartilaginous structures, such as meniscus and articular cartilage; and ligaments, such as anterior cruciate ligaments; and tendons.

The term “xenograft” is synonymous with the term “heterograft” and refers to a graft transferred from an animal of one species to one of another species. Stedman's Medical Dictionary, Williams & Wilkins, Baltimore, Md. (1995). The term “xenogeneic”, as in, for example, xenogeneic soft tissue refers to soft tissue transferred from an animal of one species to one of another species. Id.

The term “cellular disruption” as in, for example, cellular disruption treatment refers to a treatment for killing cells.

The term “portion” as in, for example, a portion of submucosa refers to all or less than all of the respective submucosa.

Once implanted in an individual, a xenograft provokes immunogenic reactions such as chronic and hyperacute rejection of the xenograft. The term “chronic rejection”, as used herein refers to an immunological reaction in an individual against a xenograft being implanted into the individual. Typically, chronic rejection is mediated by the interaction of IgG natural antibodies in the serum of the individual receiving the xenograft and carbohydrate moieties expressed on cells, and/or cellular matrices and/or extracellular components of the xenograft. For example, transplantation of cartilage xenografts from non-primate mammals (e.g., porcine or bovine origin) into humans is primarily prevented by the interaction between the IgG natural anti-Gal antibody present in the serum of humans with the carbohydrate structure Gal 1-3Gal 1-4G1cNAc-R (-galactosyl or -gal epitope) expressed in the xenograft. K. R. Stone et al., Porcine and bovine cartilage transplants in cynomolgus monkey: I. A model for chronic xenograft rejection, 63 Transplantation 640-645 (1997); U. Galili et al., Porcine and bovine cartilage transplants in cynomolgus monkey: II. Changes in anti-Gal response during chronic rejection, 63 Transplantation 646-651 (1997). In chronic rejection, the immune system typically responds within one to two weeks of implantation of the xenograft. In contrast with “chronic rejection”, “hyper acute rejection” as used herein, refers to the immunological reaction in an individual against a xenograft being implanted into the individual, where the rejection is typically mediated by the interaction of IgM natural antibodies in the serum of the individual receiving the xenograft and carbohydrate moieties expressed on cells. This interaction activates the complement system causing lysis of the vascular bed and stoppage of blood flow in the receiving individual within minutes to two to three hours.

The term “extracellular components” as used herein refers to any extracellular water, collagen and elastic fibers, proteoglycans, fibronectin, elastin, and other glycoproteins.

Source of Submucosal Xenograft Material. The xenografts of the invention can be prepared from submucosal tissue, from organs such as small intestines, stomach, and urinary bladder. The submucosa may be harvested from any non-human animal, especially a warm blooded animal, such as pigs, sheep, and cattle. The submucosa from transgenic non-human animals or from genetically altered non-human animals may also be used as xenografts.

In one embodiment, the xenograft can be formed from a segment of large or small intestinal tissue of a warm-blooded vertebrate (bird or mammal). The xenograft material contains the tunica submucosa, the muscularis mucosa and the stratum compactum of the tunica mucosa, with the tunica submucosa, muscularis mucosa and stratum compactum being delaminated from the tunica muscularis and the luminal portions of the tunica mucosa of the segment of intestinal tissue. For small intestines submucosa (“SIS”), the tri-layer intestinal segments used to form the xenografts of the invention can be used in their delaminate tubular form or they can be cut longitudinally or laterally to form elongated tissue segments. In either form, such segments have an intermediate portion and opposite end positions and opposite lateral portions which can be formed for surgical attachment to existing physiological structures, using surgically acceptable techniques. The small intestine, prior to its manipulation (delamination) as described herein to yield xenograft material, is made up of a number of discrete tissue layers. In a preferred embodiment of this invention, the tissue graft material comprises submucosa tissue and basilar mucosa tissue delaminated from a segment of the small intestine, more preferably the jejunum, a division of the small intestine extending between the duodenum and the ileum.

This material has good mechanical strength characteristics. Because the xenografts used in orthopedic surgical applications are typically placed under tension in their surgical installation, it may be preferable to combine two or even more tissue segments to provide a multi-ply (multi-layered) graft construct.

When a segment of intestine is first harvested and delaminated as described, for example, in U.S. Pat. Nos. 5,372,821 and 4,902,508 to obtain the submucosa, the xenograft material will be a tubular segment having an intermediate portion and opposite end portions. The end portions can then be formed, manipulated or shaped for attachment in a manner that will reduce the possibility of graft tearing at the point of attachment. The xenograft material can have a longitudinal dimension corresponding to the length of the segment of intestinal tissue and a lateral dimension proportioned to the diameter of the segment of intestinal tissue. The resulting segment is typically preconditioned by stretching longitudinally to a length longer than the length of the intestinal tissue segment from which it was formed. For example, the segment can be conditioned by suspending a weight from the segment, for a period of time sufficient to allow about 10 to about 20% elongation of the tissue segment. Optionally, the xenograft material can be preconditioned by stretching in the lateral dimension, because the xenograft material exhibits similar viscoelastic properties in the longitudinal and lateral dimensions. The xenograft segment is then formed in a variety of shapes and configurations, for example, to serve as a ligament or tendon replacement or substitute or a patch for a broken or severed tendon or ligament. Moreover, the segment can be shaped and formed to have a layered or even a multilayered configuration with at least the opposite end portions and/or opposite lateral portions being formed to have multiple layers of the graft material to provide reinforcement for attachment to physiological structures, including bone, tendon, ligament, cartilage and muscle.

A perforated unitary multi-laminar SIS xenograft can be prepared as described in U.S. Pat. Nos. 5,968,096 or 5,955,110. The method comprises overlapping strips of submucosal tissue, treated as described herein, with other strips of treated submucosal tissue, compressing at least the overlapped portions of the strips between two surfaces under conditions that allow or promote dehydration of the compressed submucosal sheets, and perforating the resulting unitary tissue graft construct.

In another embodiment, the xenograft of the invention can be formed from stomach submucosal tissue delaminated from both the luminal portion of the tunica mucosa and the smooth muscle layers of the muscularis externa of a stomach of a warm blooded vertebrate, as described in U.S. Pat. No. 6, 099,567.

In yet another embodiment, the xenograft of the invention can be formed from can be formed from urinary bladder submucosal tissue from a warm blooded vertebrate, as described in U.S. Pat. Nos. 5,762,966 and 5,554,389 (bladder submucosal tissue delaminated from abluminal muscle layers and at least the luminal portion of the tunica mucosa of a segment of vertebrate urinary bladder).

Methods of manufacture. To make a xenograft, the submucosal material is subjected to a cellular disruption treatment to kill the cells of the submucosa prior to in vitro digestion of the xenograft with glycosidases. The xenograft is then subjected to a digestion of the xenograft with glycosidases to remove carbohydrate moieties from the xenograft.

First, an intact small intestines is removed from a non-human animal. Harvesting of the small intestines should occur as soon as possible after slaughter of the animal (such as pig, sheep, or cattle) and preferably should be performed in the cold, i.e., in the approximate range of about 5° C. to about 20° C., to minimize enzymatic degradation of the soft tissue. The submucosa is harvested in the cold, under strict sterile technique. The xenograft is then washed in about ten volumes of sterile cold water to remove residual blood proteins and water-soluble materials. The xenograft is then immersed in alcohol at room temperature for about five minutes, to sterilize the tissue and to remove non-collagenous materials. Alternatively, the xenograft of the invention is subjected to freeze/thaw cycling as discussed above to disrupt, i.e., to kill the cells of the soft tissue.

Typically after surface carbohydrate moieties have been removed from nucleated cells and the extracellular matrix, nucleated, i.e., living cells reexpress the surface carbohydrate moieties, which can provoke continued immunogenic rejection of the xenograft. By contrast, dead cells, are unable to reexpress surface carbohydrate moieties, so that subsequent removal of antigenic surface carbohydrate moieties from the non-nucleated cells and extracellular components of a xenograft substantially permanently eliminates antigenic surface carbohydrate moieties as a source of immunogenic rejection of the xenograft.

The xenograft is then subjected to in vitro digestion with glycosidases, and specifically galactosidases, such as -galactosidase, to enzymatically eliminate antigenic surface carbohydrate moieties. In particular, -gal epitopes are eliminated by enzymatic treatment with -galactosidases, as shown in the following reaction:

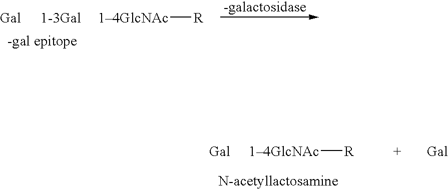

The N-acetyllactosamine residues are epitopes that are normally expressed on human and mammalian cells and thus are not immunogenic. The in vitro digestion of the xenograft with glycosidases can be accomplished by various methods. For example, the xenograft can be soaked or incubated in a buffer solution containing glycosidase. Alternatively, a buffer solution containing the glycosidase can be forced under pressure into the xenograft via a pulsatile lavage process.

Elimination of the -gal epitopes from the xenograft diminishes the immune response against the xenograft. The -gal epitope is expressed in non-primate mammals and in New World monkeys (monkeys of South America) as 1×10⁶-35×10⁶ epitopes per cell, as well as on macromolecules such as proteoglycans of the extracellular components. U. Galili et al., Man, apes, and Old World monkeys differ from other mammals in the expression of -galactosyl epitopes on nucleated cells, 263 J. Biol. Chem. 17755 (1988). This epitope is absent in Old World primates (monkeys of Asia and Africa and apes) and humans, however. Id. Anti-Gal is produced in humans and primates as a result of an immune response to -gal epitope carbohydrate structures on gastrointestinal bacteria. U. Galili et al., Interaction between human natural anti-galactosyl immunoglobulin G and bacteria of the human flora, 56 Infect. Imun. 1730 (1988); R. M. Hamadeh et al., Human natural anti-Gal IgG regulates alternative complement pathway activation on bacterial surfaces, 89 J. Clin. Invest. 1223 (1992). Since non-primate mammals produce -gal epitopes, xenotransplantation of xenografts from these mammals into primates results in rejection because of primate anti-Gal binding to these epitopes on the xenograft. The binding results in the destruction of the xenograft by complement fixation and by antibody dependent cell cytotoxicity. U. Galili et al., Interaction of the natural anti-Gal antibody with-galactosyl epitopes: A major obstacle for xenotransplantation in humans, 14 Immunology Today 480 (1993); M. Sandrin et al., Anti-pig IgM antibodies in human serum react predominantly with Gal 1-3Gal epitopes, 90 Proc. Natl. Acad. Sci. USA 11391 (1993); H. Good et al., Identification of carbohydrate structures which bind human anti-porcine antibodies: implications for discordant grafting in man. 24 Transplant. Proc. 559 (1992); B. H. Collins et al., Cardiac xenografts between primate species provide evidence for the importance of the -galactosyl determinant in hyperacute rejection, 154 J. Immunol. 5500 (1995). Furthermore, xenotransplantation results in major activation of the immune system to produce increased amounts of high affinity anti-Gal. Accordingly, the substantial elimination of -gal epitopes from cells and from extracellular components of the xenograft, and the prevention of reexpression of cellular -gal epitopes can diminish the immune response against the xenograft associated with anti-Gal antibody binding with -gal epitopes.

In additional to the treatment described above, additional steps can be taken. The xenograft may be subjected to at least one of the following treatments: radiation treatment, treatment with alcohol, ozonation, one or more cycles of freezing and thawing, and/or treatment with a chemical cross-linking agent. When more than one of these treatments is applied to the xenograft, the treatments may occur in any order.

In one embodiment, the xenografts may be treated with polyethylene glycol (PEG) prior to or concurrently with treatment with glycosidase. PEG acts as a carrier for the glycosidase by covalently bonding to the enzyme and to the collagen extracellular components. Further, PEG-treated xenografts have reduced immunogenicity.

In another embodiment, the outer surface of the xenograft (e.g., the outer lateral surface of meniscus soft tissue xenografts) optionally may be pierced to increase permeability to agents used to render the xenograft substantially non-immunogenic. A sterile surgical needle such as an 18-gauge needle may be used to perform this piercing step, or, alternatively a comb-like apparatus containing a plurality of needles may be used. The piercing may be performed with various patterns, and with various pierce-to-pierce spacings, in order to establish a desired access to the interior of the xenograft. Piercing may also be performed with a laser. In one form of the invention, one or more straight lines of punctures about three millimeters apart are established circumferentially in the outer lateral surface of the xenograft.

In yet another embodiment, the xenograft of the invention may be treated with limited digestion by proteolytic enzymes such as ficin or trypsin to increase tissue flexibility, or coated with anticalcification agents, antithrombotic coatings, antibiotics, growth factors, or other drugs which may enhance the incorporation of the xenograft into the recipient joint. The SIS xenograft of the invention may be further sterilized using known methods, for example, with additional glutaraldehyde or formaldehyde treatment, ethylene oxide sterilization, propylene oxide sterilization, or the like. The xenograft may be stored frozen until required for use.

In still another embodiment, the submucosal xenograft materials may be chemically treated to reduce immunogenicity prior to implantation into a recipient. For example, glutaraldehyde is used to cross-link or “tan” xenograft tissue in order to reduce its antigenicity, as described in detail in U.S. Pat. No. 4,755,593. Other agents such as aliphatic and aromatic diamine compounds may provide additional crosslinking through the side chain carboxyl groups of aspartic and glutamic acid residues of the collagen polypeptide. Glutaraldehyde and diamine tanning also increases the stability of the xenograft tissue. In yet a further embodiment, the xenograft may optionally be exposed to a chemical agent to tan or crosslink the proteins within the extracellular components, to further diminish or reduce the immunogenic determinants present in the xenograft. Any tanning or crosslinking agent may be used for this treatment, and more than one crosslinking step may be performed or more than one crosslinking agent may be used in order to ensure complete crosslinking and thus optimally reduce the immunogenicity of the xenograft. For example, aldehydes such as glutaraldehyde, formaldehyde, adipic dialdehyde, and the like, may be used to crosslink the extracellular collagen of the xenograft in accordance with the method of the invention. Other suitable crosslinking agents include aliphatic and aromatic diamines, carbodiimides, diisocyanates, and the like.

When glutaraldehyde is used as the crosslinking agent, for example, the xenograft may be placed in a buffered solution containing about 0.05 to about 5.0% glutaraldehyde and having a pH of about 7.4. Any suitable buffer may be used, such as phosphate buffered saline or trishydroxymethylaminomethane, and the like, so long as it is possible to maintain control over the pH of the solution for the duration of the crosslinking reaction, which may be from one to fourteen days, and preferably from three to five days.

Alternatively, the xenograft can be exposed to a crosslinking agent in a vapor form, including, but not limited to, a vaporized aldehyde crosslinking agent, such as, for example, vaporized formaldehyde. The vaporized crosslinking agent can have a concentration and a pH and the xenograft can be exposed to the vaporized crosslinking agent for a period of time suitable to permit the crosslinking reaction to occur. For example, the xenograft can be exposed to vaporized crosslinking agent having a concentration of about 0.05 to about 5.0% and a pH of about 7.4, for a period of time which can be from one to fourteen days, and preferably from three to five days. Exposure to vaporized crosslinking agent can result in reduced residual chemicals in the xenograft from the crosslinking agent exposure.

The crosslinking reaction continues until the immunogenic determinants are substantially removed from the xenogeneic soft tissue, but the reaction iserminated prior to significant alterations of the mechanical properties of the xenograft When diamines are also used as crosslinking agents, the glutaraldehyde crosslinking occurs after the diamine crosslinking. After the crosslinking reactions have proceeded to completion, the xenograft is rinsed to remove residual chemicals, and 0.01-0.05 M glycine may be added to react with any unreacted aldehyde groups that remain.

In an alternative embodiment, the xenograft may be treated by exposure to ultraviolet radiation for about fifteen minutes or gamma radiation in an amount of about 0.2 to 3 MegaRad. Such radiation sterilizes the xenograft.

In another embodiment, the xenograft may be treated by again being placed in an alcohol solution. Any alcohol solution may be used to perform this treatment. Preferably, the xenograft is placed in a 70% solution of isopropanol at room temperature.

In another embodiment, the xenograft may be subjected to ozonation.

In another embodiment, the xenograft may be treated by freeze/thaw cycling. For example, the xenograft may be frozen using any method of freezing, so long as the xenograft is completely frozen, i.e., no interior warm spots remain which contain unfrozen soft tissue. Preferably, the xenograft is dipped into liquid nitrogen for about five minutes to perform this step of the method. More preferably, the xenograft is frozen slowly by placing it in a freezer. In the next step of the freeze/thaw cycling treatment, the xenograft is thawed by immersion in an isotonic saline bath at room temperature (about 25° C.) for about ten minutes. No external heat or radiation source is used, in order to minimize fiber degradation.

The submucosal xenograft tissues may also be subjected to various physical treatments in preparation for implantation. For example, U.S. Pat. No. 4,755,593 discloses subjecting xenograft tissue to mechanical strain by stretching to produce a thinner and stiffer biomaterial for grafting. Tissue for allograft transplantation is commonly cryopreserved to optimize cell viability during storage, as disclosed, for example, in U.S. Pat. No. 5,071,741; U.S. Pat. No. 5,131,850; U.S. Pat. No. 5,160,313; and U.S. Pat No. 5,171,660. U.S. Pat. No. 5,071,741 discloses that freezing tissues causes mechanical injuries to cells therein because of extracellular or intracellular ice crystal formation and osmotic dehydration.

Accordingly, the submucosal xenograft produced in accordance with the method of the invention is substantially non-immunogenic for implantation into humans, while generally maintaining the mechanical properties of a native soft tissue.

Methods of use. The xenograft of the invention, or a segment or portion thereof, may be implanted into damaged human joints by those of skill in the art using known arthroscopic surgical techniques. Specific instruments for performing arthroscopic techniques are known to those of skill in the art, which ensure accurate and reproducible placement of soft tissue implants.

Once the xenograft is placed within a body, it aids in the proliferation of new blood vessels, which is important for the wound-healing process. The blood vessels nourish the graft and supply vital molecules that the body needs to rebuild the damaged tissue. The material also strengthens in response to stress, much like natural soft tissue.

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The following EXAMPLE is presented in order to more fully illustrate the preferred embodiments of the invention. This EXAMPLE should in no way be construed as limiting the scope of the invention, as defined by the appended claims.

### EXAMPLE 1

### Assessment of Primate Response to Implanted SIS Xenograft Treated with -Galactosidase

In this EXAMPLE, porcine SIS implants are treated with -galactosidase to eliminate -galactosyl epitopes, the implants are transplanted into cynomolgus monkeys, and the primate response to the soft tissue implants is assessed.

Porcine SIS xenografts are sterilely prepared. Each SIS specimen is carefully identified and dissected free of adhering tissue, thereby forming the xenograft. The SIS xenografts are washed for at least five minutes with an alcohol, such as ethanol or isopropanol, to remove fluid and lipid soluble contaminants. The SIS xenografts are then frozen at a temperature ranging from about −35° C. to about −90° C., and preferably at a temperature up to about −70° C.

Each SIS xenograft specimen is cut into two portions. Each first portion is immersed in a buffer solution containing -galactosidase at a predetermined concentration. The specimens are allowed to incubate in the buffer solutions for a predetermined time period at a predetermined temperature. Each second portion is incubated under similar conditions as the corresponding first portion in a buffer solution in the absence of -galactosidase and serves as the control.

At the end of the incubation, the SIS xenografts are washed under conditions that allow the enzyme to diffuse out. Assays are performed to confirm the complete removal of the -gal epitopes.

Each SIS xenograft is implanted in the supra patellar pouch of six cynomolgus monkeys. With the animals under general inhalation anesthesia, an incision of about 1 cm is made directly into the supra patellar pouch at the superior medial border of the patella extending proximally. A piece of the porcine SIS xenograft of about 0.5 cm to about 1 cm in length is placed into the pouch with a single 3-0 nylon stitch as a marking tag.

SIS xenografts are also implanted in six cynomolgus monkeys using the following implantation procedure. With the animals under general inhalation anesthesia, the anatomic insertion sites for the xenografts are identified and drilled to accommodate a substantially 9 mm in diameter by 40 mm in length bone plug. The xenograft is brought through the drill holes and affixed with interference screws.

The implantation procedures are performed under sterile surgical technique, and the wounds are closed with 3-0 vicryl or a suitable equivalent known to those of ordinary skill in the art. The animals are permitted unrestricted cage activity and monitored for any sign of discomfort, swelling, infection, or rejection. Blood samples (e.g., 2 ml) are drawn periodically (e.g., every two weeks) for monitoring of antibodies.

The occurrence of an immune response against the xenograft is assessed by determining anti-Gal and non-anti-Gal anti-soft tissue antibodies (i.e., antibodies binding to soft tissue antigens other than the -gal epitopes) in serum samples from the transplanted monkeys. At least two ml blood samples are drawn from the transplanted monkeys on the day of implant surgery and at periodic (e.g., two week) intervals post-transplantation. The blood samples are centriged and the serum samples are frozen and evaluated for the anti-Gal and other antibody activity.

Anti-Gal activity is determined in the serum samples in ELISA with -gal-BSA as solid phase antigen, according to methods known in the prior art, such as, for example, the methods described in Galili et al., Porcine and bovine cartilage transplants in cynomolgus monkey. II Changes in anti-Gal response during chronic rejection, 63 Transplantation 645-651 (1997).

Assays are conducted to determine whether -galactosidase treated xenografts induce the formation of anti-soft tissue antibodies. For measuring anti-soft tissue antibody activity, ELISA assays are performed according to methods known in the prior art, such as, for example, the methods described in K. R. Stone et al., Porcine and bovine cartilage transplants in cynomolgus monkey: I. A model for chronic xenograft rejection, 63 Transplantation 640-645 (1997).

The xenograft specimens are optionally explanted at one to two months post-transplantation, sectioned and stained for histological evaluation of inflammatory infiltrates. Post-transplantation changes in anti-Gal and other anti-cartilage soft tissue antibody activities are correlated with the inflammatory histologic characteristics (i.e., granulocytes or mononuclear cell infiltrates) within the xenograft, one to two months post-transplantation, using methods known in the art, as, for example, the methods described in K. R. Stone et al., Porcine and bovine cartilage transplants in cynomolgus monkey: I. A model for chronic xenograft rejection, 63 Transplantation 640-645 (1997).

The animals that have had xenograft implantations are allowed to recover and are monitored closely until the incisions have healed and the gait is normal. The xenograft samples are collected, processed, and examined microscopically.

Portions of the xenograft implants and surrounding tissues are frozen in embedding mediums for frozen tissue specimens in embedding molds for immunohistochemistry evaluation according to the methods known in the prior art. “TISSUE-TEK®” O.C.T. compound which includes about 10% w/w polyvinyl alcohol, about 4% w/w polyethylene glycol, and about 86% w/w nonreactive ingredients, and is manufactured by Sakura FinTek (Torrence, Calif., USA) is a non-limiting example of a possible embedding medium for use with the present invention. Other embedding mediums known to those of ordinary skill in the art may also be used. The remaining implant and surrounding tissue is collected in 10% neutral buffered formalin for histopathologic examination.

The foregoing description has been presented only for the purposes of illustration and is not intended to limit the invention to the precise form disclosed, but by the claims appended hereto.

What is claimed is:

1. A method of preparing a submucosal xenograft for implantation into a human, which comprises the steps of:
(a) removing at least a portion of a submucosal from a non-human animal to provide a xenograft;
(b) washing the xenograft in water and alcohol;
(c) subjecting the xenograft to a cellular disruption treatment; and
(d) digesting the xenograft with a glycosidase to remove substantially a plurality of surface carbohydrate moieties from the xenograft;
whereby the xenograft is substantially non-immunogenic and has substantially the same mechanical properties as a native soft tissue.

2. The method of claim 1, wherein the cellular disruption treatment comprises freeze/thaw cycling.

3. The method of claim 1, wherein the cellular disruption treatment comprises exposure to gamma radiation.

4. The method of claim 1, wherein the glycosidase is a galactosidase.

5. The method of claim 4, wherein the galactosidase is an α-galactosidase.

6. The method of claim 1, further comprising the step of
(e) depleting substantially a plurality of proteoglycans from the xenograft.

7. The method of claim 6, wherein the depleting step comprises digesting the xenograft with at least one proteoglycan-depleting factor selected from the group consisting of chondroitinase ABC, hyaluronidase, chondroitin AC II lyase, keratanase, trypsin and fibronectin fragment.

8. The method claim 1 further comprising the step of:
following step (c), piercing the xenograft.

9. The method of claim 1, further comprising the step of:
following step (c), treating the xenograft with at least one enzyme.

10. The method of claim 9, wherein the enzyme is selected from the group consisting of ficin and trypsin.

11. The method of claim 1 further comprising the step of:
following step (c), treating the xenograft with one or more agents selected from the group consisting of anticalcification agents, antithrombotic agents, antibiotics, and growth factors.

12. The method of claim 1 further comprising the step of:
following step (c), sterilizing the xenograft.

13. The method of claim 12, wherein the sterilizing step comprises sterilizing the xenograft with one or more agents selected from the group consisting of ethylene oxide, and propylene oxide.

14. The method of claim 1, further comprising the step of:
following step (c), treating the xenograft with at least one crosslinking agent.

15. The method of claim 14, wherein the crosslinking agent is selected from the group consisting of aldehydes, aromatic diamines, carbodiimides, and diisocyanates.

16. The method of claim 15 wherein at least one crosslinking agent is glutaraldehyde.

17. The method of claim 16 wherein the xenograft is crosslinked using a solution containing about 0.01 percent to about 5 percent glutaraldehyde.

19. The method of claim 15 wherein the crosslinking agent in a vapor form.

20. The method of claim 1 further comprising the step of:
following step (c), treating the xenograft with polyethylene glycol.

21. An article of manufacture comprising a substantially non-immunogenic submucosal xenograft for implantation in to a human, produced by:
(a) removing at least a portion of a submucosa from a non-human animal to provide a xenograft;
(b) washing the xenograft in water and alcohol;
(c) subjecting the xenograft to a cellular disruption treatment; and
(d) digesting the xenograft with a glycosidase to remove substantially a plurality of surface carbohydrate moieties from the xenograft;
whereby the xenograft is substantially non-immunogenic and has substantially the same mechanical properties as a corresponding portion of a submucosal tissue.

22. The article of manufacture of claim 21 wherein the xenograft has further been after step (c), digested with a proteoglycan-depleting factor to remove substantially a plurality of proteoglycans from the xenograft,

23. The article of manufacture of claim 21, wherein the xenograft has a plurality of punctures into the xenograft for increasing permeability to agents and enzymes.

24. The article of manufacture of claim 21, wherein the xenograft has been sterilized.

25. The article of manufacture of claim 21, wherein the xenograft has been crosslinked.

26. A submucosal xenograft tissue for implantation into a human, comprising a portion of submucosal tissue from a non-human animal,
(a) wherein the portion includes a plurality of extracellular components and only dead cells, the extracellular components and the dead cells having substantially no surface -galactosyl moieties
(b) whereby the portion of the submucosal xenograft tissue is substantially non-immunogenic in a primate.

27. The xenograft of claim 26 wherein at least a portion of the xenograft tissue is excised from the jejunum of a warm-blooded vertebrate.

28. The xenograft of claim 26 wherein the portion is the tunica submucosa, the muscularis mucosa and the stratum compactum of the tunical mucosa, of a segment of small intestine.

29. The xenograft of claim 26 wherein said tunica submucosa, muscularis mucosa and stratum compactum are delaminated from the tunica muscularis and the luminal portion of the tunica mucosa of said section of small intestine.

30. The xenograft of claim 26, wherein the portion of the ligament has a second block of bone affixed to a second end thereof opposite the first end.

31. A submucosal xenograft for implantation into a human comprising a portion of a submucosa from a non-human animal,
(a) wherein the portion includes a plurality of extracellular components, a plurality of substantially only dead cells, the extracellular components having reduced proteoglycans, and an aldehyde in an amount ranging from about 0.01 percent to about 5 percent crosslinking a plurality of proteins of the extracellular components, the dead cells and extracellular components having substantially no surface carbohydrate moieties which are susceptible to glycosidase digestion, and
(b) whereby the portion of the submucosa is substantially non-immunogenic and has substantially the same mechanical properties as the native soft tissue.

32. The xenograft of claim 31, wherein the extracellular components and the substantially only dead cells have substantially no surface α-galactosyl moieties.

33. The xenograft of claim 31, wherein the xenograft has a plurality of punctures.

34. A method of preparing a submucosal xenograft for implantation into a human, which comprises the steps of:
(a) removing at least a portion of submucosa from a non-human animal to provide a xenograft;
(b) washing the xenograft in water and alcohol;
(c) subjecting the xenograft to a cellular disruption treatment; and
(d) depleting substantially a plurality of proteoglycans from the xenograft,
whereby the xenograft is substantially non-immunogenic and has substantially the same mechanical properties as the native soft tissue.

35. The method of claim 34, further comprising the step of:
following step (c), sterilizing the xenograft.

36. The method of claim 34, wherein the sterilizing step comprises sterilizing the xenograft with one or more agents selected from the group consisting of ethylene oxide and propylene oxide.

37. The method of claim 34, further comprising the step of:
after step (d), removing substantially a plurality of surface carbohydrate moieties from the xenograft.

38. The method of claim 34, wherein the depleting step comprises digesting the xenograft with a glycosidase.

39. The method of claim 34, wherein the glycosidase is a galactosidase.

40. The method of claim 34, wherein the galactosidase is an α-galactosidase.

41. A submucosal xenograft for implantation into a human comprising a portion of a submucosa from a non-human animal,
(a) wherein the portion includes only dead cells and a plurality of extracellular components, the extracellular components having reduced proteoglycans,
(b) whereby the portion is substantially non-immunogenic and has substantially the same mechanical properties as the native soft tissue.

42. The xenograft of claim 41, wherein the extracellular components and the substantially only dead cells have substantially no surface -galactosyl moieties.
